(19) United States
(12) Patent Application Publication
(10) Pub. No.: US 2007/0167823 A1
(43) Pub. Date: Jul. 19, 2007

(54) Imaging catheter and method for volumetric ultrasound

(75) Inventors: Warren Lee, Clifton Park, NY (US); Kenneth Wayne Rigby, Clifton Park, NY (US)

Correspondence Address: General Electric Company;GEGR Patent Docket Rm., Bldg. K-1 Rm. 4A59, One Research Circle, Niskayuna, NY, 12309, US

(73) Assignee: General Electric Company

(21) Appl. No.: 11/314,338

(22) Filed: Dec. 20, 2005

(51) Int. Cl. A61B 8/14

(52) U.S. Cl. 600/463

(57) ABSTRACT

An imaging catheter assembly and method for use in volumetric ultrasound imaging and catheter-guided procedures are provided. The imaging catheter assembly comprises a transducer array for acquiring image data at a given image plane and a motion controller coupled to the transducer array for translating the transducer array along a direction perpendicular to a direction of the image plane in order to image a three-dimensional (3D) volume.

### BACKGROUND

The invention relates generally to an imaging catheter, and more particularly to a transducer array assembly for use in volumetric ultrasound imaging and catheter-guided treatment such as cardiac interventional procedures.

In ultrasound imaging, spatial resolution is a critical factor in image quality. For severely space-constrained applications, such as imaging from catheter-mounted transducers (intracardiac echocardiography or ICE), two major limitations prevent the acquisition of high quality real-time three-dimensional (RT3D) volumetric images, generally referred to as volumes. The first limitation is due to the number of signal conductors that can physically fit within the limited size of the catheter. This limitation is especially severe for two-dimensional arrays, which can be electronically scanned in two dimensions to form RT3D volumes, because they typically require M×N connections, where M and N are the number of rows and columns of transducer elements in the array, respectively. The second limitation is due to the small physical size available for the acoustic aperture. Transducers confined to this spatial extent typically generate ultrasound beams that diverge rapidly with distance, resulting in poor spatial resolution. The poor resolution in turn hinders the clinician's ability to identify important anatomical and physiological targets.

The issue of acquiring real-time three-dimensional volumes has been addressed with the advent of two-dimensional array transducers, however, as discussed above, it is difficult to generate images with sufficient field of view and resolution. Mechanically scanning one-dimensional (1D) transducer arrays currently exist, but have only been applied to much larger abdominal probes, where space constraints are not as severe.

Clinical applications such as cardiac interventional procedures would benefit from catheters capable of acquiring three-dimensional (3D) volumes. For example, cardiac interventional procedures such as the ablation of atrial fibrillation are complicated due to the lack of an efficient method to visualize the cardiac anatomy in real-time. Intracardiac echocardiography (ICE) has recently gained interest as a potential method to visualize interventional devices as well as cardiac anatomy in real-time. Current commercially available catheter-based intracardiac probes used for clinical ultrasound B-scan imaging have limitations associated with the monoplanar nature of the B-scan images. RT3D imaging may overcome these limitations. Existing 1D catheter transducers have been used to make 3D ICE images by rotating the entire catheter, but the resulting images are not real-time. Other available RT3D ICE catheters use a two-dimensional (2D) array transducer to steer and focus the ultrasound beam over a pyramidal-shaped volume. However, many challenges exist with 2D arrays, such as low sensitivity due to the small element size, and increases in system cost and complexity.

Currently, in order to obtain useful 3D volumes along a subject's anatomical tract, e.g. a vascular structure or other cavity, a catheter containing for example a 1D array with multiple elements oriented along the catheter's long axis is inserted into the region of interest and manually rotated to obtain image data along the desired anatomical tract. The single-plane image produced by the 1D array is thereby rotated so that a pyramidal 3D image volume is acquired. Such a catheter is shown in FIG. 1. As shown in FIG. 1, transducer 11 includes a plurality of transducer elements 13 oriented along the catheter's long axis. Using such rotation methods necessitate manual control and user intervention to facilitate movement through the anatomical tract.

Therefore, as intracardiac interventional procedures are more commonly used, there is a need to overcome the problems described above. Further, there is a need to enable improved 3D volumetric intracardiac imaging and interventional procedures along an anatomical tract, particularly where there are space constraints.

### BRIEF DESCRIPTION

In a first aspect of the invention, an imaging catheter assembly for use in volumetric ultrasound imaging and catheter-guided procedures is provided. The imaging catheter assembly comprises a transducer array for acquiring image data at a given image plane and a motion controller coupled to the transducer array for translating the transducer array along a direction perpendicular to a direction of the image plane in order to image a 3D volume.

In a second aspect of the invention, a method for volumetric imaging and catheter-guided procedures is provided. The method comprises obtaining image data for a 3D volume using an imaging catheter. The imaging catheter assembly comprises a transducer array for acquiring image data at a given image plane and a motion controller coupled to the transducer array for translating the transducer array along a direction perpendicular to the image plane in order to image a 3D volume.

### DRAWINGS

These and other features, aspects, and advantages of the present invention will become better understood when the following detailed description is read with reference to the accompanying drawings in which like characters represent like parts throughout the drawings, wherein:

FIG. 1 is an illustration of a catheter assembly according to prior art;

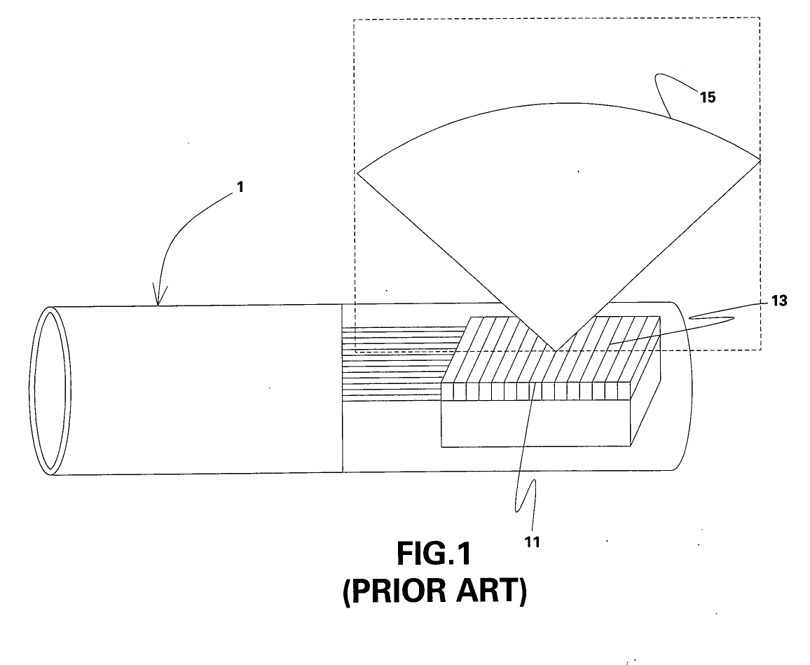

FIG. 2 is a block diagram of an exemplary ultrasound imaging and therapy system, in accordance with aspects of the present technique;

FIG. 3 is a side and internal view of an embodiment of a catheter assembly having a single linear transducer array assembly for use in the imaging system of FIG. 2;

FIG. 4 is an illustration of another embodiment of a catheter assembly having multiple transducer array assemblies for use in the imaging system of FIG. 2;

FIG. 5 is a side and internal view of another embodiment and corresponding operation of a catheter assembly having a cylindrical transducer array for use in the imaging system of FIG. 2;

FIG. 6 is an illustration of another embodiment of a catheter assembly having multiple cylindrical transducer array assemblies; and,

FIG. 7 is an illustration of another embodiment of a catheter assembly having transducer arrays comprising electrostrictive ceramic or micromachined ultrasound transducer (MUT) arrays for use in the imaging system of FIG. 2.

### DETAILED DESCRIPTION

As will be described in detail hereinafter, a catheter assembly in accordance with exemplary aspects of the present technique is presented. Based on image data acquired by the catheter assembly, a three-dimensional volume of an anatomical region may be imaged and diagnostic information and/or the need for therapy in the anatomical region may be obtained.

In accordance with aspects of the present invention, the aforementioned limitations are overcome by using a transducer array that acquires image data at a given image plane and the transducer array is translated mechanically, or the active portion of the transducer is array is translated electronically, in a direction perpendicular to the image plane in order to image a 3D volume. The elements of the transducer array are electronically phased in order to acquire a sector image perpendicular to the long axis of the catheter, and the array is translated along the catheter axis in order to acquire the three-dimensional volume through assembly of two-dimensional images. Thus, problems associated with 2D arrays such as sensitivity and system cost and complexity are avoided using this method. It is to be appreciated that transducer arrays other than 1D arrays may be used, but then complexity and space constraints are added.

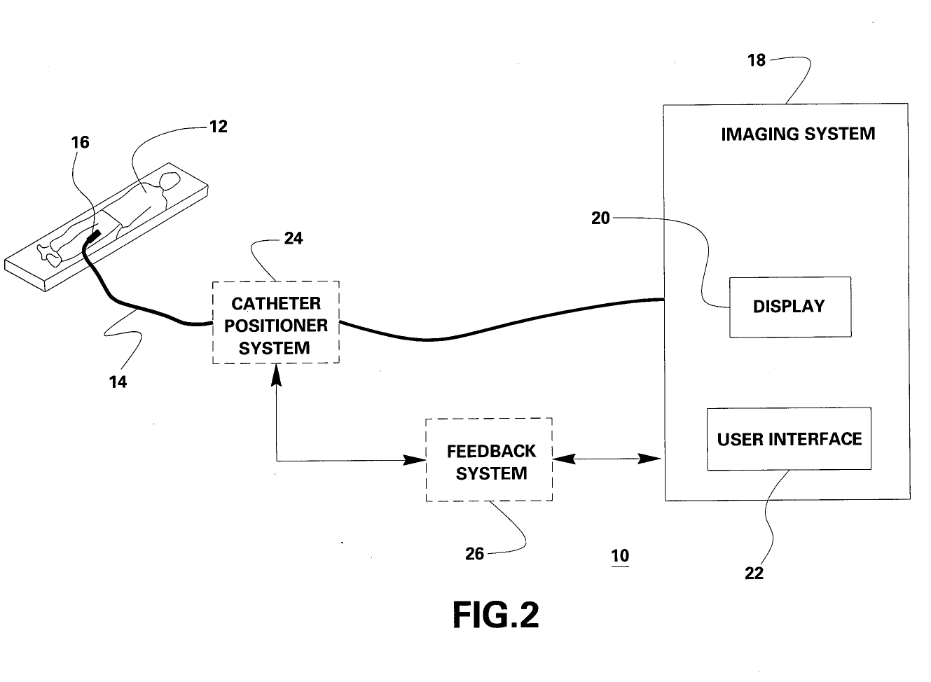

FIG. 2 is a block diagram of an exemplary system 10 for use in imaging and providing therapy to one or more regions of interest in accordance with aspects of the present technique. The system 10 may be configured to acquire image data from a patient 12 via a catheter 14. As used herein, “catheter” is broadly used to include conventional catheters, transducers, probes or devices adapted for imaging as well as adapted for applying therapy. Further, as used herein, “imaging” is broadly used to include two-dimensional imaging, three-dimensional imaging, or preferably, real-time three-dimensional imaging. Reference numeral 16 is representative of a portion of the catheter 14 disposed inside the vasculature of the patient 12.

In accordance with aspects of the present technique, the catheter 14 may be configured to image an anatomical region to facilitate assessing the need for therapy in one or more regions of interest within the anatomical region of the patient 12 being imaged. Additionally, the catheter 14 may also be configured to deliver therapy to the identified one or more regions of interest, such as including a delivery port (not shown) or therapeutic devices (not shown) in the catheter. As used herein, “therapy” is representative of ablation, percutaneous ethanol injection (PEI), cryotherapy, and laser-induced thermotherapy. Additionally, “therapy” may also include delivery of tools, such as needles for delivering gene therapy, for example. Additionally, as used herein, “delivering” may include various means of providing therapy to the one or more regions of interest, such as conveying therapy to the one or more regions of interest or directing therapy towards the one or more regions of interest. As will be appreciated, in certain embodiments the delivery of therapy, such as RF ablation, may necessitate physical contact with the one or more regions of interest requiring therapy. However, in certain other embodiments, the delivery of therapy, such as high intensity focused ultrasound (HIFU) energy, may not require physical contact with the one or more regions of interest requiring therapy.

The system 10 may also include a medical imaging system 18 that is in operative association with the catheter 14 and configured to image one or more regions of interest. The imaging system 10 may also be configured to provide feedback for therapy delivered by the catheter or separate therapy device (not shown). In addition, the medical imaging system 18 may be configured to acquire image data representative of the anatomical region of the patient 12 via the catheter 14.

As illustrated in FIG.2, the imaging system 18 may include a display area 20 and a user interface area 22. However, in certain embodiments, such as in a touch screen, the display area 20 and the user interface area 22 may overlap. Also, in some embodiments, the display area 20 and the user interface area 22 may include a common area. In accordance with aspects of the present technique, the display area 20 of the medical imaging system 18 may be configured to display an image generated by the medical imaging system 18 based on the image data acquired via the catheter 14. Additionally, the display area 20 may be configured to aid the user in defining and visualizing a user-defined therapy or surgical pathway. It should be noted that the display area 20 may include a three-dimensional display area. In one embodiment, the three-dimensional display may be configured to aid in identifying and visualizing three-dimensional shapes.

Further, the user interface area 22 of the medical imaging system 18 may include a human interface device (not shown) configured to facilitate the user in identifying the one or more regions of interest for delivering therapy (for example) using the image of the anatomical region displayed on the display area 20. The human interface device may include a mouse-type device, a trackball, a joystick, a stylus, or a touch screen configured to facilitate the user to identify the one or more regions of interest for display on the display area 20.

It may be noted that although the exemplary embodiments illustrated hereinafter are described in the context of an ultrasound system, other medical imaging systems such as, but not limited to, optical imaging systems are also contemplated for catheter-guided imaging applications, particularly in space confined applications.

As depicted in FIG. 2, the system 10 may include an optional catheter positioning system 24 configured to reposition the catheter 14 within the patient 12 in response to input from the user. Moreover, the system 10 may also include an optional feedback system 26 that is in operative association with the catheter positioning system 24 and the medical imaging system 18. The feedback system 26 may be configured to facilitate communication between the catheter positioning system 24 and the medical imaging system 18.

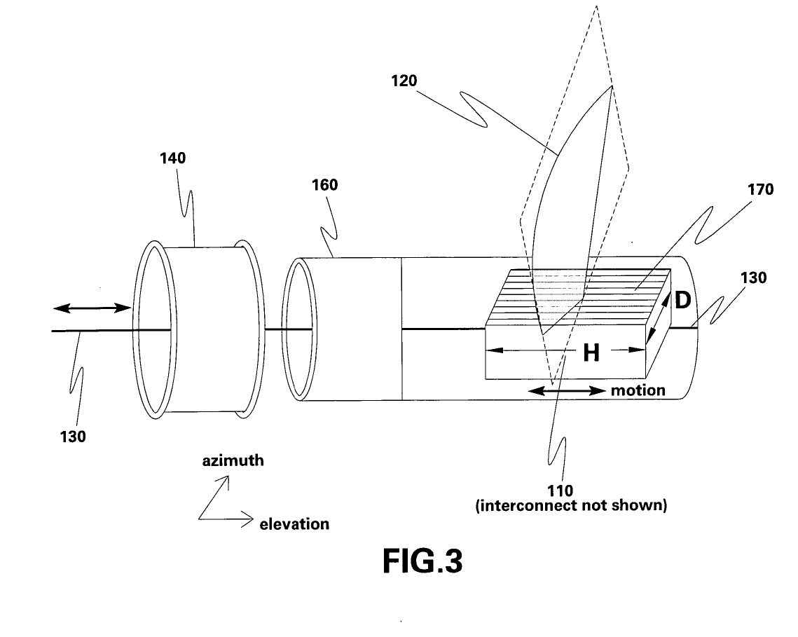

Referring to FIG. 3, an embodiment of a catheter assembly 14 for use in the imaging system of FIG. 2 is shown. As shown, the catheter assembly 14 comprises a transducer array 110, a motion guide 130 and actuator 140. The assembly 14 further includes a catheter housing 160 for enclosing the transducer array 110 and motion guide 130. Actuator 140 is shown external to catheter housing 160, however in an alternative embodiment actuator 140 may be internal to catheter housing. It is to be appreciated that actuators and motor controllers are becoming available in miniaturized configurations that may be applicable to embodiments of the present invention. Also, catheter housing 160 also encloses cables and other connections (not shown) coupled between the transducer array 110 and the imaging system shown in FIG. 2 for use in receiving/transmitting signals between the transducer and the imaging system. In an embodiment, transducer array 110 is a high-frequency, e.g. about 7 MHz to about 40 MHz, 1D array transducer having multiple elements 170 arranged in the azimuth dimension (perpendicular to the longitudinal axis of the catheter). In accordance with configurations of catheter 160 and applications for use described herein, the applications of such a catheter necessitate relatively small sizes, for example a catheter diameter of about 1 to about 4 mm. Thus, aperture size will also be relatively small which results in a rapid divergence of the ultrasound beam. In accordance with embodiments for transducer array 110, transducer elements 170 are arranged along a direction perpendicular to the catheter axis. The total width D of the elements spans most of the catheter diameter, which maximizes the resolution of the resulting beam. In addition, the transducer may operate at a high frequency to compensate for the loss in resolution due to the small size of the aperture. As shown in FIG. 3, transducer elements 170 are arranged along the short axis of the catheter (shown as azimuth direction), i.e. the diameter of catheter 160. In embodiments, increasing height H of transducer elements 170 has the effect of increasing sensitivity and far-field resolution of the transducer array. The transducer itself may be made of a variety of materials, including, but not limited to, PZT, micromachined ultrasound transducers (MUTs), or polyvinyldene fluoride (PVDF). It should be noted that although the embodiments illustrated are described in the context of a catheter-based transducer, other types of transducers such as transesophageal transducers or transthoracic transducers are also contemplated. The ultrasound beam is electronically steered in the azimuth dimension (perpendicular to the catheter long axis), creating a single-plane B-scan image 120. The transducer array 110 is attached to the motion guide 130, and actuator 140 is configured for moving the motion guide and transducer in the elevation dimension (perpendicular to the image plane and parallel to the longitudinal axis of the catheter ). Imaged plane 120 is shown as an exemplary 2D image from a B-scan. The catheter further includes a fluid-filled acoustic window (not shown) to allow coupling of acoustic energy from the transducer array 110 to the region or medium of interest.

Referring further to FIG. 3, in operation, catheter assembly comprises a one-dimensional transducer array 110 that acquires image data at a given image plane, e.g. the azimuth direction, and the transducer array 110 is then translated along motion guide 130 in a direction perpendicular to the image plane (elevation direction, which corresponds to the longitudinal axis of the catheter) direction in order to image a 3D volume. In this embodiment, transducer array 110 is mechanically moved by the actuator 140 in combination with the motion guide 130 in the elevation dimension, with B-scans 120 being acquired at each elevational location. The B-scans 120 are assembled to form a 3D volume.

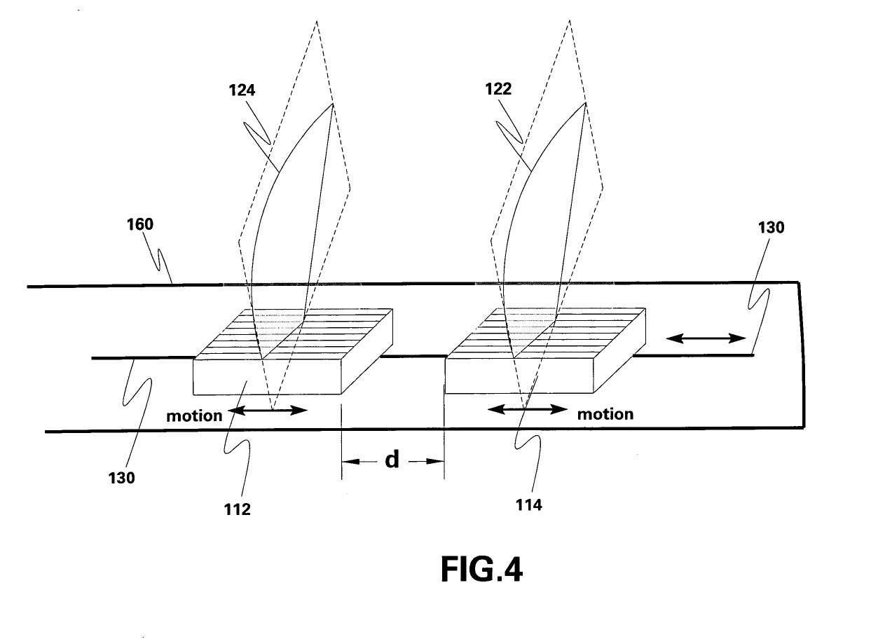

Referring now to FIG. 4, an alternative embodiment for a transducer array is shown. In this embodiment, transducer array comprises multiple 1D arrays (112, 114) spaced apart by a distance d and fixed to the motion guide 130, reducing the amount of linear motion necessary to acquire the 3D volume and allowing for an increased scanning rate. In an embodiment, an imaging scan sequence involving multiple arrays may comprise array 114 being activated first and acquiring image plane 122. Array 112 is then activated, acquiring image plane 124. The motion guide 130 is moved a certain amount and the sequence is repeated at a plurality of positions along the motion guide 130 until the image planes form a continuous volume. In another embodiment, arrays 112 and 114 are simultaneously activated, thus improving the acquisition speed for a given volume size.

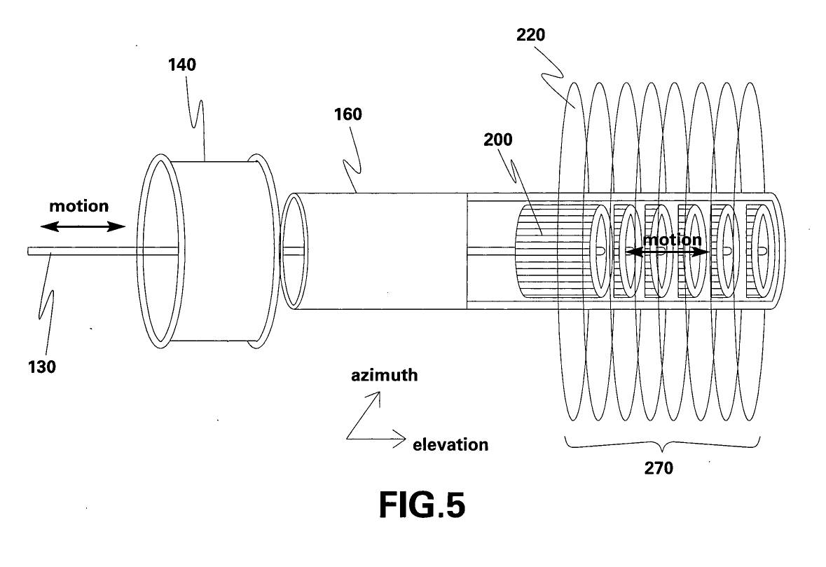

Referring to FIG. 5, an alternative embodiment for a transducer array is shown. In this embodiment, transducer array comprises a single cylindrical array 200 that is electronically scanned to form a circular planar image in the azimuth dimension. Analogous to the operation described with reference to FIG. 3, the cylindrical array 200 is moved along the motion guide 130 in the elevation dimension. At each elevation location, a circular image 220 is generated, and thus a cylindrical volume 270 is acquired.

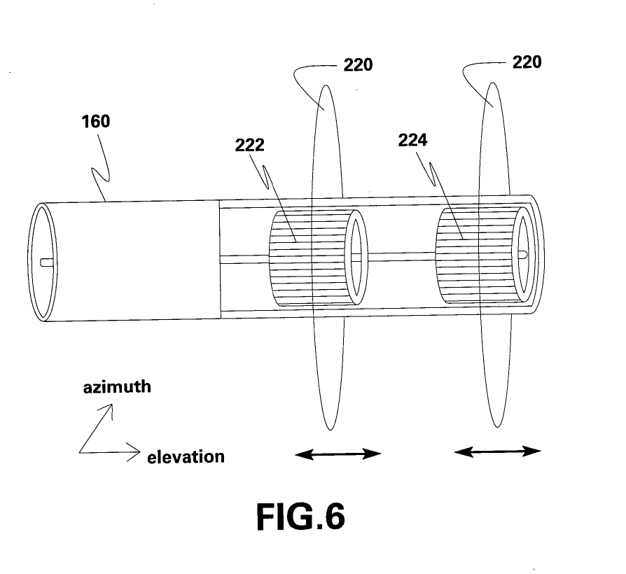

FIG. 6 shows a further alternative embodiment using multiple cylindrical arrays 222 and 224 fixed to the motion guide 130 and operable as described with reference to FIG. 4 having multiple arrays in order to reduce the amount of linear motion necessary to acquire the 3D cylindrical volume. In the embodiments shown in FIG. 5 and 6, cylindrical arrays may include full cylinders, partial cylinders or curved transducer arrays. Operation would be similar to that described with reference to FIG. 3 and 4.

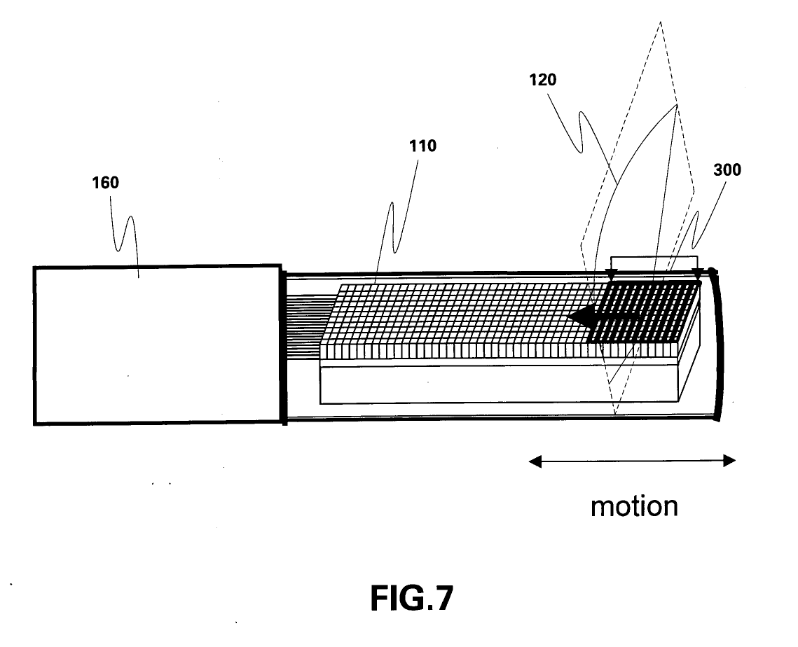

In a further embodiment, a method for volumetric ultrasound imaging and catheter-guided procedures comprises using embodiments of catheter assembly as described herein in which a transducer array is used for acquiring image data at a given image plane and a motion controller coupled to the transducer array is used for translating the transducer array along a direction perpendicular to a direction of the image plane in order to image a three-dimensional (3D) volume. In operation using the embodiments of a transducer array as described with reference to FIG. 3-6, the amount of translation of the transducer array(s) determines the field of view, or size of the three-dimensional volume acquired along the elevation dimension. The larger the translation, the larger the acquired volume. In certain cases, it may be desirable to reduce the amount of translation that is required, yet sustain a large field of view. In such a situation, the use of multiple transducer arrays that are spaced a certain distance apart in the elevation dimension and the arrays are electronically activated in an alternating sequence, so that at each translational position, images are acquired at the elevational position of each array. The reduction in translation that is achieved is equivalent to the number of arrays that are used. In a further embodiment, the multiple arrays may be simultaneously activated, thus decreasing the time required to acquire data from a given volume. In a further embodiment, the method of imaging comprises electrical control of the translation of the transducer array, which will be described in greater detail with reference to FIG. 7.

Motion control system has two main components: linear actuator 140 and motion guide 130 which serves to transmit the motion of the actuator to the transducer array. Motion control may be electronic or mechanical. Motion guide 130 may be made up of a material that is bendable, yet stiff with respect to its long axis. Actuator 140 is a linear actuating mechanism that may reside inside the space-constrained environment (i.e. in the catheter housing 160), or it may be external to the space-constrained environment, with its motion transmitted to the array through the guide. The ability to place the actuator 140 outside the space constrained environment is significant, due to the limited variety and power of actuating mechanisms available that fit inside very small medical devices such as catheters.

Referring to FIG. 7, an alternative embodiment for catheter assembly comprises transducer arrays that are electrostrictive or micromachined ultrasound transducer (MUT) arrays, wherein such transducer arrays have activatable regions 300 for acquiring image data 120 at a given image plane. In this embodiment, motion controller comprises bias control electronics which select the active regions of the transducer. Therefore, a three-dimensional data set may acquired by assembling the electronically scanned two-dimensional images. Alternatively, motion controller comprises a layer of bias control electronics 310 s used to control the location of the active transducer region within each row. By assembling the image planes 120, a 3D volume is acquired. Further in these embodiments, the transducer consists of elements arranged in rows and columns. The row dimension is the catheter long axis and the column dimension is perpendicular to the catheter axis. Each row of transducer elements is connected to a single system channel. A layer of bias control electronics 310 is used to activate columns of the elements, thus controlling the location of the active transducer region. The active region may be electronically translated along the catheter long axis using bias control electronics. At each active region location, an image plane is acquired. By assembling the image planes, a 3D volume is acquired. Electronic scanning through the use of bias controlled electrostrictive ceramic or MUT arrays allows the fast acquisition of three-dimensional data without requiring moving parts, while retaining the simplicity associated with only requiring a limited number of system channels.

In accordance with aspects of the present invention, the embodiments described herein overcome the two major limitations preventing the generation of high spatial resolution 3D ultrasound images from within a space-constrained environment. The first limitation is the small size of the aperture, which causes a rapid divergence of the ultrasound beam. This limitation may be compensated for by the high frequency operation of the transducer array. Further, the elevation dimension of the transducer array and corresponding transducer elements may be selected to increase sensitivity and elevation resolution of the transducer array. The second limitation is the number of signal conductors that can physically fit inside the catheter. The catheter cannot accommodate enough conductors for a high-resolution, high-frequency 2D array transducer. This limitation is bypassed with this invention due to the fact that the transducer array for this invention is a linear array, rather than a two-dimensional array—therefore, only M connections are required, rather than N×M, where N is the number of columns in the transducer and M is the number of rows. Further, in embodiments, the number of signal channels may be reduced to correspond to the number of transducer elements oriented along a short axis of the catheter. The 1D array provides a high-quality image in a single plane and translating the array (or the active aperture) moves the image plane to sweep out and image a 3D volume.

While only certain features of the invention have been illustrated and described herein, many modifications and changes will occur to those skilled in the art. It is, therefore, to be understood that the appended claims are intended to cover all such modifications and changes as fall within the true spirit of the invention.

1. An imaging catheter assembly for use in volumetric ultrasound imaging and catheter-guided procedures, the assembly comprising:
a transducer array used for acquiring image data at a given image plane;
a motion controller coupled to the transducer array for translating the transducer array along a direction perpendicular to a direction of the image plane in order to image a three-dimensional (3D) volume.

2. The imaging catheter assembly of claim 1 wherein the transducer array comprises a linear one-dimensional (1D) transducer array.

3. The imaging catheter assembly of claim 1 wherein the wherein the motion controller is configured to translate the transducer array at a plurality of positions and the transducer array acquires a plurality of image planes along the 3D volume.

4. The imaging catheter of claim 3 wherein the plurality of image planes are combined to obtain the volumetric image data.

5. The imaging catheter assembly of claim 3 wherein the motion controller comprises:
an actuator; and,
a motion guide coupled to the actuator and the transducer array;
wherein the actuator and motion guide cooperate to translate the transducer array.

6. The imaging catheter assembly of claim 1 wherein the transducer array comprises at least two transducer arrays spaced apart along an axis of the catheter and wherein each of the transducer array is translated along a portion of the axis of the cathether and wherein each transducer acquires image data of the 3D volume along its respective portion of the axis.

7. The imaging catheter assembly of claim 1 wherein the transducer array comprises a cylindrical transducer array having transducer elements arranged perpendicular to an axis of the catheter.

8. The imaging catheter assembly of claim 7 wherein the cylindrical transducer comprises at least one of a full cylinder, a partial cylinder or a curved transducer array

9. The imaging catheter assembly of claim 7 wherein the transducer array comprises at least two cylindrical transducer arrays having transducer elements arranged perpendicular to an axis of the catheter and the two cylindrical transducer arrays are spaced apart along an axis of the catheter and wherein each of the cylindrical transducer arrays is translated along a portion of the axis of the catheter and wherein each transducer acquires image data of the 3D volume along its respective portion of the axis.

10. The imaging catheter assembly of claim 1 wherein the transducer array comprises at least one of an electrostrictive or a micromachined transducer (MUT) transducer array and wherein the transducer array comprises activatable regions for acquiring image data at a given image plane.

11. The imaging cathether assembly of claim 10 wherein the motion controller comprises a bias control electronics circuit to electronically activate activatable regions of the transducer to acquire image data along the 3D volume.

12. The imaging catheter assembly of claim 1 wherein the transducer comprises transducer elements arranged in a direction perpendicular to a long axis of the catheter.

13. A method for performing volumetric ultrasound imaging and catheter-guided procedures, the method comprising:
obtaining image data for a three-dimensional (3D) volume using an imaging catheter, wherein the imaging catheter comprises:
a transducer array used for acquiring image data at a given image plane;
a motion controller coupled to the transducer array for translating the transducer array along a direction perpendicular to a direction of the image plane in order to image the three-dimensional (3D) volume.

14. The method of claim 13 wherein the transducer array comprises a linear one-dimensional (1D) transducer array.

15. The method of claim 13 wherein the wherein the motion controller is configured to translate the transducer array at a plurality of positions and the transducer array acquires a plurality of image planes along the 3D volume.

16. The method of claim 15 wherein the plurality of image planes are combined to obtain the volumetric image data.

17. The method of claim 15 wherein the motion controller comprises:
an actuator; and,
a motion guide coupled to the actuator and the transducer array;
wherein the actuator and motion guide cooperate to translate the transducer array.

18. The method of claim 13 wherein the transducer array comprises at least two transducer arrays spaced apart along an axis of the cathether and wherein each of the transducer array is translated along a portion of the axis of the cathether and wherein each transducer acquires image data of the 3D volume along its respective portion of the axis.

19. The method of claim 13 wherein the transducer array comprises a cylindrical transducer array having transducer elements arranged perpendicular to an axis of the catheter.

20. The method of claim 19 wherein the transducer array comprises at least two cylindrical transducer arrays having transducer elements arranged perpendicular to an axis of the catheter and the two transducer arrays are spaced apart along an axis of the cathether and wherein each of the transducer array is translated along a portion of the axis of the cathether and wherein each transducer acquires image data of the 3D volume along its respective portion of the axis.

21. The method of claim 13 wherein the transducer array comprises at least one of an electrostrictive or a micromachined transducer (MUT) transducer array and wherein the transducer array comprises activatable regions for acquiring image data at a given image plane.

22. The method of claim 21 wherein the motion controller comprises a bias control electronics circuit to electronically activate activatable regions of the transducer to acquire image data along the 3D volume.
